(19) United States
(12) Patent Application Publication
(10) Pub. No.: US 2020/0363406 A1
(43) Pub. Date: Nov. 19, 2020

(54) HIGHLY-SPECIFIC ASSAYS

(71) Applicant: The University of Houston System, Houston, TX (US)

(72) Inventors: Hui CHEN, Houston, TX (US); Mary CRUM, Katy, TX (US); Dimple CHAVAN, Houston, TX (US); Ekaterini KOURENTZI, Bellaire, TX (US); Binh V. VU, Houston, TX (US); Richard C. WILLSON, Houston, TX (US)

(73) Assignee: The University of Houston System, Houston, TX (US)

(21) Appl. No.: 16/759,240

(22) PCT Filed: Oct. 26, 2018
(86) PCT No.: PCT/US18/57790

§ 371 Date: Apr. 24, 2020

Related U.S. Application Data
(60) Provisional application No. 62/577,523, filed on Oct. 26, 2017.

(51) Int. Cl. G01N 33/542

(52) U.S. Cl. CPC G01N 33/542; G01N 2458/10

(57) ABSTRACT

Assay compositions and methods for detection of analytes that include covalent modification of assay elements, such that they are preserved, destroyed, created, or immobilized. Methods for detecting an analyte in a biological sample. The method includes providing a mixture of a biological sample potentially containing the analyte, and a molecular recognition element physically coupled to a covalent modification agent, wherein the molecular recognition element is capable of specific recognition of the analyte, and exposing the mixture to a first set of reaction conditions, wherein the analyte and molecular recognition element can associate to form a recognition complex. Upon formation of the recognition complex, the method further includes generating by use of the covalent modification agent, a template complex; and exposing the template complex to a second set of reaction conditions, wherein the template complex is amplified to generate a detectable product indicative of the presence of the analyte.

### CROSS-REFERENCE TO RELATED APPLICATIONS

This application claims the benefit of priority of U.S. Provisional Application No. 62/577,523, filed Oct. 26, 2017, the disclosure of which is hereby incorporated by reference in its entirety.

### TECHNICAL FIELD

The present disclosure relates to compositions and methods for implementing highly-specific assays. More specifically, certain compositions provided herein include covalent modification agents physically associated with molecular recognition elements that are used to detect analytes in an ultra-sensitive and quantitative manner.

### BACKGROUND

Tests, assays and diagnostics based on molecular recognition are widely used in a variety of settings. A tradeoff is often observed between sensitivity of detection, cost, convenience and specificity. Polymerase chain reaction has been a revolutionary technology for detection of nucleic acids but there are no robust amplification techniques for proteins and other non-nucleic acid molecules. Immunoassay labels such as enzymes and fluors cannot achieve sufficient sensitivity for tiny biopsy samples, low-level pathogens, and trace contaminants and biomarkers. Immuno-PCR uses DNA labels to report antibody binding but because the sensitivity of PCR greatly penalizes any non-specific binding, the technique is not widely adopted. The levels of specificity and detection sensitivity achievable using current amplification and detection methods are limited, especially under conditions of low analyte concentration or higher background noise. Improved methods are desired to overcome these limitations.

### SUMMARY

Disclosed herein are compounds and methods addressing the shortcomings of the art, which may provide any number of additional or alternative advantages, including very sensitive detection with low non-specific background. New assays and diagnostic methods are disclosed herein using a covalent modification agent (CMA) to preserve, immobilize, liberate, create or destroy a highly-detectable component, conditioned upon the presence of an analyte recognized by a molecular recognition element associated to the CMA. Methods disclosed herein combine antibody specificity and PCR detectability in a way that circumvents problems of false positives and high background noise.

Disclosed herein are methods for detecting an analyte in a biological sample. The method includes providing a mixture of a biological sample potentially containing the analyte, and a molecular recognition element directly or indirectly physically coupled to a covalent modification agent, wherein the molecular recognition element is capable of specific recognition of the analyte, and exposing the mixture to a first set of reaction conditions, wherein the analyte and molecular recognition element can associate to form a recognition complex. Upon formation of the recognition complex, the method further includes generating by use of the covalent modification agent, a template complex; and exposing the template complex to a second set of reaction conditions, wherein the template complex is amplified to generate a highly-detectable component indicative of the presence of the analyte.

The covalent modification agent can be a helicase and the template complex is a single stranded DNA. The covalent modification agent can be a DNA polymerase and the template complex is a double stranded DNA product. The covalent modification agent can be a DNA glycosylase and the template complex is a double stranded DNA product. The covalent modification agent can be a DNA ligase and the template complex is a ligated double stranded DNA product formed from two or more oligonucleotides present in the second set of reaction conditions. The highly-detectable component is formed by amplification of the ligated double stranded DNA product. The covalent modification agent can be an enzyme or metal or organometallic catalyst on an inorganic support, or coupled to a protein or nucleic acid, and the highly-detectable component can be an enzyme cofactor, and aptamer, a volatile compound, or a compound with high detectability by chromatography, mass spectrometry, or differential or ion mobility spectrometry. The covalent modification agent can be a ubiquitin protein ligase and, in some embodiments, it is physically coupled to the molecular recognition element via a cleavable linker.

In some embodiments, the highly-detectable component is a nucleic acid or enzyme. In certain embodiments, the nucleic acid is modified by a CMA, which can be a nuclease, kinase, methylase, polymerase, phosphatase, RNase H, esterase, exonuclease, or ligase. In certain embodiments, detection includes an amplification method such as PCR, RPA, HDA, NASBA, LAMP, PLA, RCA, SDA, MDA, or quantitative, digital or competitive versions of amplification methods. In some embodiments, detection includes fluorescence, phosphorescence, chemiluminescence, imaging, absorbance, scattering, conductivity cytometry, chromatography, lateral-flow assay, differential mobility analysis, droplet analysis or catalytic activity.

In some embodiments, the molecular recognition element can be a nucleic acid, aptamer, virus, peptide, nanobody, lectins, sugar or antibody. In some embodiments, the CMAs may be modified or associated with molecular recognition elements, such as DNA or RNA probes, lectins, viruses, phage, cells, antibodies, Fab fragments, affitins, nanobodies, or aptamers. In some embodiments, the molecular recognition elements may be covalently attached to CMAs, e.g. by NHS/EDC chemistry, sulfhydryl/maleimide chemistry, genetic fusion, or click chemistry. In some embodiments, the molecular recognition elements may be non-covalently attached to CMAs, such as by physical adsorption or biotin/avidin linkage. In some embodiments, the CMAs and/or molecular recognition elements of the present disclosure may be associated with each other, or with cells, viruses or particles. Attachment may be by linkers, such as triethoxysilylbutyraldehyde (TESBA), poly(ethylene glycol) (PEG), (3-aminopropyl)triethoxysilane (APTES), alkanes, trialkoxysilanes and the like.

### BRIEF DESCRIPTION OF THE DRAWINGS

The present disclosure can be better understood by referring to the following figures. The emphasis is placed upon illustrating the principles of the disclosure and not limited to the assay components and methods provided.

FIG. 1 is a graphical representation of an assay using streptavidin-coated-magnetic-nanoparticle-based NIP (SAMNP-NIP) with ELISA for the detection of human chorionic gonadotropin (hCG).

FIG. 2 is a schematic of nanoparticle-based proximity ligation assay (NP-PLA). As shown in (A), in traditional Immuno-PCR (iPCR), the PCR template oligo is directly used as the reporter. Non-specifically bound oligos are PCR-amplifiable giving rise to an increased nonspecific background signal. As shown in (B), in nanoparticle-based proximity ligation assay (NP-PLA), avidin-coated nanoparticles serve as the reporters. The avidin nanoparticles bring the two split parts (biotinylated oligo-A and biotinylated oligo-B) of the PCR template and the biotinylated bridge oligo-C into proximity. Then oligo-A and oligo-B are ligated by DNA ligase and the resulting oligomer serves as the PCR template. Any non-specifically bound oligos in NP-PLA cannot be ligated into a PCR-amplifiable template and thus the non-specific background signal is significantly decreased.

FIG. 3 shows the comparison of the detectability of different nanoparticles with proximity ligation assay (PLA). (Top, left to right): (A) streptavidin-coated gold nanoparticles (GNP), (B) streptavidin-coated magnetic nanoparticles (MNP) and (C) ANANAS nanoparticles. (Bottom) (D) Dose response curves for the three different nanoparticles in PLA; and (E) Dose-response curves in hCG immunoassays using MNP and ANANAS particles as antibody labels. The −Delta Ct values were calculated by subtracting the Ct value of samples from the Ct value of the blank control; the mean±standard deviation; n=3.

FIG. 4 shows a graphical representation of detection of human chorionic gonadotropin (hCG) using nanoparticle-based proximity ligation assay (NP-PLA), immuno-PCR (iPCR) and enzyme-linked immunosorbent assay (ELISA). The −delta Ct values are calculated by subtracting the Ct value of samples from the Ct value of the blank control. Mean±standard deviation; n=6.

FIG. 5 shows a graphical representation of the use of melting peak based competitive PCR (mp-cPCR) to quantify the results of the nanoparticle-based proximity ligation assay (NP-PLA) for the detection of human chorionic gonadotropin (hCG). (A) Comparison of the results of NP-PLA for hCG detection with realtime PCR and cPCR. Mean±standard deviation; n=6. (B, top) Melting curves of 6 independent blank samples in the cPCR based NP-PLA for hCG detection. (B, bottom) Melting curves of 6 independent samples with 0.1 pg/mL hCG in the cPCR-based NP-PLA for hCG detection.

FIG. 6 is Table of a set of optimized oligos used for PLA testing.

FIG. 7 is Table showing optimization of the assay conditions for the detection of hCG with NP-PLA.

FIG. 8 is a Table showing the use of melting peak based competitive PCT (mp-cPCR) for quantification of the nanoparticle based proximity ligation assay (NP-PLA) for the detection of human chorionic gonadotropin (hCG).

FIG. 9 is an image detailing the working principle of competitive polymerase chain reaction (cPCR).

FIG. 10 is an image detailing the principle of melting peak-based competitive polymerase chain reaction (mp-cPCR).

FIG. 11 is an image of quantification of the ligation yield on the surface of nanoparticles. (A) Standard curve of the synthetic full-length PCR template spiked in 10 μL ligation buffer and mixed with 10 μL PCR master mix. PCR was run in the same settings as described in Methods. (B) Ligated PCR template per MNP detected by PCR at different particle counts. Mean±standard deviation; n=3.

FIG. 12 is a graphical representation of the limit of detection for human chorionic gonadotropin (hCG) using nanoparticle-based proximity ligation assay (NP-PLA). The dash line indicates the level of the blank signal plus 3 standard deviations of the blank signal. The gray squares represent the individual signals of sextuplicates. The crosses represent the mean values. The two-tailed P value equals 0.0027.

FIG. 13 is an image showing the process of constructing plasmids with several copies of a target sequence, as was used in preparation of certain DNA-Avidin nanoparticles described herein.

FIG. 14 is a graphical representation of the analysis of DNA-Avidin nanoparticle with four copies of template using NanoSight.

FIG. 15 is a graphical representation of Detection of hCG using DNA-Avidin nanoparticle with 1 copy of template using QPCR.

FIG. 16 is a graphical representation of Detection of hCG using DNA-Avidin nanoparticle with 4 copy of template using QPCR.

FIG. 17 is a graphical representation of the fluorescence of heat treated ANANAS nanoparticles.

### DETAILED DESCRIPTION

Reference will now be made to the exemplary embodiments illustrated in the specific examples and drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise. Parameters disclosed herein (e.g., temperature, time, concentrations, etc.) may be approximate.

The section headings used herein are for organizational purposes and are not to be construed as limiting the subject matter described.

### Analytes of Interest

The methods and compositions disclosed herein may be utilized to detect various analytes of interest from various specimens. For instance, in some embodiments, analytes of interest include, without limitation, nucleic acids such as genomic DNA, methylated DNA, specific methylated DNA sequences, messenger RNA, fragmented DNA, chromosomal DNA, mitochondrial DNA, fetal DNA, fetal RNA, rDNA, cDNA, fragmented RNA, fragmented mRNA, rRNA, viral RNA, siRNA, microRNA, SSU RNAs, LSU-rRNAs, 5S rRNA, spacer region DNA from rRNA gene clusters, 5.8S rRNA, 4.5S rRNA, 10S RNA, RNAseP RNA, guide RNA, telomerase RNA, snRNAs—e.g. U1 RNA, scRNAs, exosomes, polymerase chain reaction (PCR) products, cpDNA, artificial RNA, plasmid DNA, oligonucleotides, polyA mRNA, RNA/DNA hybrid, pathogen DNA, pathogen RNA, replication protein A (RPA) amplification product, loop-mediated isothermal amplification product (LAMP), restriction fragments, YAC, BAC, cosmid, metabolite, metabolic intermediate, interleukins, hormones (such as insulin, testosterone, and HCG), organelle, biomarker, lipid, carbohydrate, pathogen carbohydrate or protein, human protein, markers of genetic engineering, glycoprotein, lipoprotein, phosphoprotein, specific phosphorylated or acetylated variant of a protein, or viral coat proteins, cell surface receptor, peptides, drugs, spores, enzyme substrate, enzyme, and enzyme reaction product, anthrax spore, teichoic acid, prion, chemical toxins (such as pesticides, herbicides, atrazine, PCBs and digoxin), and other chemical warfare agent, biological warfare agent, or pollutant.

### Analyte Source

Analytes to be detected or quantified may be isolated from various sources. For instance, in some embodiments, analytes may be isolated from cells, tissues, or body fluids, such as a biopsy specimen, blood, serum, plasma, stool, saliva, sweat, sputum, vomit, CSF, lavage fluid, tears, ocular fluids, transcellular fluid, urethral or genital secretions, exudate from lesions or areas of inflammation, nasal wash, nasal swab, throat swab, urine, hair, cell lysate, circulating tumor cells, exosomes, FNAB cells, FACS fraction, immunomagnetic isolate, air filtrate, FFPE slices, fresh-frozen specimens, fresh tissue, frozen tissue, neutral formalin-treated tissue, a formalin fixed paraffin embedded tissue block, an ethanol-fixed paraffin-embedded tissue block, surgical site, FACS-sorted population, laser-capture microdissection fraction, magnetic separation subpopulation, FFPE extracts. In some embodiments, analytes may be obtained from environmental samples from the soil, air, or water, agricultural products (grains, seeds, plants, meat, livestock, vegetables, rumen contents, milk, etc.); contaminated liquids; surface scrapings or swabbings; biofilms, cell cultures, pharmaceutical production cultures, CHO cell cultures, bacterial cultures, virus-infected cultures, microbial colonies, and combinations thereof. In certain embodiments, the sample source is obtained from a crime scene or related to a criminal act and as such the sample source is used for various forensic purposes. Such samples can include bodily fluids such as blood, saliva, sweat, serum, plasma, stool, sputum, vomit, urine, tears, semen, etc., other human or animal samples such as hair and hair roots, and other types of evidence such as beverage samples (e.g. alcohol), clothing or fibers, illegal drugs or pharmaceuticals, and explosive compositions.

In some embodiments the analyte to be detected may be obtained from surfaces or components of clothing, shoes, garments, personal protective gear and personal equipment, or other gear, bathrooms, military settings, equipment or objects in the vicinity of or near a facility for the production of agricultural or food products. In some embodiments the analyte to be detected may be obtained from any surfaces or components of objects suspected of contamination with illicit substances or hazardous materials or analytes associated with the production of illicit substances or hazardous materials.

### Sample Preparation

In various embodiments, the assays can include one or more sample-preparation steps. In some embodiments, the sample preparation steps may utilize various sample preparation agents. In some embodiments, sample preparation may include, without limitation, concentrating, enriching, and/or partially purifying the analytes of interest. For instance, in some embodiments, the samples may be pre-treated by centrifugation, sedimentation, fractionation, field-flow fractionation, elutriation, monolithic separation, extraction, adsorption, protease, nuclease, dialysis, osmosis, buffer exchange, partitioning, washing, de-waxing, leaching, lysis, osmolysis, amplification, denature/renaturation, crystallization, freezing, thawing, cooling/heating, degasification, sonication, pressurization, drying, magnetophoresis, electrophoresis, dielectrophoresis, acoustophoresis, precipitation, microencapsulation, sterilization, autoclaving, germination, culturing, PCR, disintegration of tissue, extraction from FFPE, LAMP, NASBA, emulsion PCR, phenol extraction, silica adsorption, immobilized metal affinity chromatography (IMAC), filtration, affinity capture, capture from a large volume of a dilute liquid source, air filtration, surgical biopsy, FNA, flow cytometry, laser capture microdissection, and combinations thereof.

In some embodiments, sample preparation may include, without limitation, use of various concentrations of a dilute species from a liquid or gaseous environment using a filter, isolation of a subset of cells from a complex blood sample, breakage of cells to liberate analytes of interest, extraction of the analyte from a solid sample, or removal of lipids and particulates, which could interfere with later analysis.

In some embodiments, sample preparation may involve amplification of the analyte to be detected. For instance, amplification may include the use of the polymerase chain reaction to amplify nucleic acids or nucleation chain reaction to amplify prion proteins, or growth of an organism. Another way to amplify the detectability of an analyte is to grow an assembly of biomolecules, such as an actin filament or immune complex. Another method is to use a nucleating agent (e.g., of bubbles, crystals or polymerization) as an element of the analyte. Where available, these methods can greatly facilitate subsequent analysis.

### Analyte Modification

The analytes of the present disclosure can be modified in various manners. In some embodiments, the analytes can be modified by labeling, conjugation, methylation, esterification, dephosphorylation, phosphorylation, hydrolysis, proteolysis, acetylation, deacetylation, methylation, demethylation, denaturation, oxidation, conjugation, haloacetic acid modification, hatching, growth, excystation, passaging, culture, de-blocking, proteolysis, nuclease digestion, cDNA preparation, amplification, DNA ball preparation, PEGylation, clonal amplification, multiplication, charge enhancement, hybridization, antibody binding, adsorption, aptamer binding, photo-linking, reduction, oxidation, and combinations thereof.

### Assay Elements

In some embodiments, the analytes of the present disclosure may be detected by assays using various elements, such as reporters or labels. The labels can also be attached to the molecular recognition elements, instead of the analytes. In some embodiments, the label elements which can be part of, all of, associated with, or attached to reporters or labels include a nanoparticle, gold particle, silver particle, silver, copper, zinc, iron, iron oxide, or other metal coating or deposit, polymer, drag tag, magnetic particle, buoyant particle, microbubble, metal particle, charged moiety, silicon dioxide, with and without impurities (e.g., quartz, glass, etc.), poly(methylmethacrylate), polyimide, silicon nitride, gold, silver, quantum dot, CdS, carbon dot, a phosphor such as silver-activated zinc sulfide or doped strontium aluminate, a fluor, a quencher, polymer, PMMA, polystyrene, retroreflector, bar-coded or labeled particle, porous particle, pellicular particle, solid particle, nanoshells, nanorods, IR absorbers, microwave absorbers, microspheres, liposomes, microspheres, polymerization initiators, photografting reagents, proteins, molecular recognition elements, linkers, self-assembled monolayers, PEG, dendrimers, charge modifiers, PEG, silane coupling agents, initiators of growth from polymer grafts from the label surface, stabilizing coatings, zwitterions, zwitterionic peptides, zwitterionic polymers, magnetic materials, magnetic materials of Curie temperature below 200° C., enzyme, microbial nanowires, DNA including aptamer sequences, phage modified for conductivity, fusions or conjugates of detectable elements with molecular recognition elements, Streptavidin, NeutrAvidin, Avidin, ExtrAvidin, or biotin-binding proteins, biotin, biotinylated molecules, biotinylated polymers, biotinylated proteins, anti-antibody aptamer, aptamer directed to antibody-binding protein, an azide or terminal alkyne or other click chemistry participant, and combinations thereof. In some embodiments, the surface of a reporter is modified by covalent attachment of molecular recognition elements. In some embodiments, the surface of a reporter is modified by adsorption of molecules for colloidal stability or molecular recognition.

In some embodiments, assay elements may be functionalized with various functional groups on their surfaces. In some embodiments, components of the assay may be coated or functionalized with moieties that reduce non-specific binding in analytical assays or tests. Exemplary functional groups include, without limitation, amine groups, carboxyl groups, aldehydes, ketones, hydroxyls, maleimide groups, sulfhydryl groups, thiols, hydrazides, anhydrides, alkenes, alkynes, azides, and combinations thereof. In other embodiments, assay elements can be coupled to aldehydes on antibodies created by oxidizing the polysaccharides on the F_{c} portion of the antibody with periodate. In further embodiments, Protein A or other proteins that bind specifically to the F_{c} portion of an antibody can be attached to an assay element, and then used to bind to an antibody in an oriented manner.

In certain embodiments, the assay elements include an antibody, an aptamer, a natural or recombinant protein, a recombinant Pleckstrin homology domain, FYVE domain, PX domain, ENTH domain, CALM domain, PDZ domains, PTB domains, FERM domain or Metallothioneins. Metallothioneins have the ability to bind to metals including arsenic, zinc, mercury, selenium, lead, iron, copper, cadmium, mercury, and silver. Because of the binding abilities of metallothioneins, they can be used in assays relating to metal pollutants in a patient and the environment.

In other embodiments, the assay elements include inositide-recognition modules (modules that specifically bind to inositol phosphates). In another embodiment, the assay elements include members of the clathrin adaptor protein and arrestin families. Clathrin adaptor proteins specifically bind certain proteins and lipids, while arrestins specifically bind G-protein coupled receptors.

In some embodiments, the assay elements of the present disclosure may be utilized in various assay settings. In some embodiments, the assay settings may include, thermal cyclers, incubators, electrochemical cells, lateral flow media, microtiter wells, surface-bound assays, flow through assays, assays associated with buoyant materials, relocation assays, nanopores, plasmonic layers with holes below 120 nm diameter for extraordinary optical transmission, microchannels, microdroplets, nanowells, or assays associated with magnetic materials for concentration or force stringency. In some embodiments, the present disclosure relates to compositions of matter comprising assay elements and porous membranes such as, but not limited to, nitrocellulose, glass fibers, and cotton fibers.

### Molecular Recognition Element (MRE)

Many analytical methods, including those of interest in the present invention, involve molecular recognition, and also transduction of the molecular recognition event into a usable signal. Molecular recognition refers to the high affinity and specific tendency of particular chemical species to associate with one another, or with organisms or viruses displaying target chemical species. Well-known examples of molecular recognition include the hybridization of complimentary DNA sequences into the famous double helix structure with very high affinity, and the recognition of foreign organisms or molecules in the blood stream by the antibodies produced by mammals, or selected analytes by deliberately selected monoclonal antibodies. There are many other examples of molecular recognition elements, including the recognition of carbohydrate molecules by lectins, nucleic acid recognition by proteins and nucleic acid analogs, the binding of analytes by antibody fragments, derivatives, and analogs, and a host of other examples.

In some embodiments, the label elements of the present disclosure may also be associated with various molecular recognition elements. In some embodiments, the molecular recognition elements may be part of, associated with, or attached to labels. In some embodiments, molecular recognition elements can include, without limitation, antibody, antibody fragment, antibody analog, affibody, camelid or shark antibody analog, nucleic acid, carbohydrate, aptamer, ligand, chelators, peptide nucleic acid, locked nucleic acid, backbone-modified nucleic acid, DARPin, molecularly imprinted polymers, lectin, padlock probe, substrate, receptor, viral protein, mixed, cDNA, metal chelate, boronate, peptide, enzyme substrate, enzyme reaction product, lipid bilayer, cell, tissue, microorganism, yeast, bacterium, parasite, protozoan, virus, antigen, hapten, biotin, hormone, drug, anti-RNA/DNA hybrid antibody, mutS, anti-DNA antibody, anti-methylation antibody, or an anti-phosphorylation antibody.

### Covalent Modification Agents

In some embodiments, the assay involves a covalent modification agent, which may be a catalytic or reactive moiety. In some embodiments, the covalent modification agent functions as a template generator element. The template generator element can be a protein, such as nucleic acid-modifying enzyme. These enzymes include without limitation a ligase, helicases, methylase, kinase, demethylase, dephosphorylase, phosphatase, RNase H, polymerase, exonuclease or endonuclease. The covalent modification agent may include an esterase or protease, biotin ligase, sumoylation enzymes, sortases, or ubiquitin ligases, lipoyl protein ligase, or lipoic acid ligase. Covalent modification agents may be associated with, expressed in, coupled to, or displayed on particles, surfaces, cells, spores, or phages. Chemical covalent modification agents include click reagents, aldehydes, cross-linkers, supported metals such as gold and platinum, and photoreactive moieties.

### Amplification or Signal Enhancement Methods

An assay includes a molecular recognition event that is detected by a usable signal. In certain embodiments, the signal may be amplified or enhanced by a signal enhancement method, which may act upon the analyte, molecular recognition element, a label associated to either one of them, or a component of such label.

Many applications discussed herein mention assays in which detection of an analyte involves direct binding of the reporter to the analyte at or in a specific region of interest and an increase in signal from the region of interest indicates a positive signal. In another embodiment, a readout method by which the analyte is detected is by determination of the presence or absence of the label in locations different from the locations expected in the absence of the analyte. Of particular interest are assays in which binding (or the suppression of binding, or competition) gives rise to the presence or absence of a signal. For instance, a phosphorescent label can be displaced from a pre-specified region by the presence of analyte, and then a decrease in luminescence from that region or an increase in luminescence elsewhere would indicate a positive signal. In some embodiments, luminescence is used to quantitatively or qualitatively obtain a signal in an assay by imaging with a film-based or digital camera (e.g., a digital camera with a CMOS, CCD or other type of sensor). In some embodiments, luminescence may be measured with a luminometer, fluorometer, PMT, avalanche photodiode, spectrophotometer, or other similar instrument capable of measuring intensity of light.

In some embodiments, the signal amplification or enhancement method may include hatching, growth, PCR, solid-phase PCR, RPA, LATE, EATL, RCA, LAMP, 3SR, LCR, SDA, MDA, HDA, or hot-restart amplification, solid-phase RCA, silver staining, metal deposition or plating, nickel, copper or zinc deposition, gold particle growth, polymerization, particle binding, grafting, photografting, click chemistry, a copper(I)-catalyzed 1,2,3-triazole forming reaction between an azide and a terminal alkyne, and combinations thereof. In an embodiment where a nucleic acid is amplified, the amplification product can comprise an aptamer such as aptamers recognizing materials such as agarose, cellulose, or nitrocellulose for self-immobilization or self-capture. In other embodiments, the amplification product includes a detectable DNA sequence such as an aptamer recognizing a reporter such as a dye, fluor or enzyme, or a catalytic DNA sequence such as a DNAzyme.

In some embodiments, a luminescence signal from a reporter such as a chemiluminescence-active enzyme, fluor, or phosphor may be read or detected by an optical sensor such as, but not limited to, a charge-coupled device (CCD) sensor, CCD image sensor, complementary metal-oxide-semiconductor (CMOS) sensor, CMOS image sensor, camera, cell phone camera, photodiode, avalanche photodiode, single-photon avalanche diode, superconducting nanowire single-photon detector, photoresistor, photomultiplier, photomultiplier tube, phototube, photoemissive cell, photoswitch, phototransistor, photonic crystal, fiber optic sensor, electro-optical sensor, luminometer, or fluorometer. In some embodiments the luminescence signal from the label elements may be read, detected, or inferred from photochemical reactions, such as those that occur in photographic film, with the photochemical reactions stimulated or initiated by luminescence from the label elements. In some embodiments the luminescence signal from the label elements may be read or detected visually by the naked eye, or with the assistance of an optical amplifier or intensifier.

In some embodiments, a cell phone, smart phone, or portable electronic device such as, but not limited to, a tablet, personal digital assistant, or laptop can be used to detect signals from reporters for qualitative or quantitative assay readout. In some embodiments, a cell phone or portable electronic device may be coupled to an attachment device to detect or test for the presence or absence of an analyte in a sample. In some embodiments, averaging techniques are used to achieve a higher signal-to-noise ratio of detection method in an assay for detecting the presence or absence of an analyte.

### Specificity Enhancement

In some embodiments, the specificity of detection of analytes may be enhanced through removal of non-specifically bound labels by chemical or physical means. In some embodiments, chemical means of removal include denaturants, temperature, acids, bases, osmolytes, surfactants, polymers, and solvents. In some embodiments, physical means of removal include force, vibration, buoyancy, washing, centrifugation, sedimentation field-flow, magnetic force, electrophoretic force, dielectrophoretic force, sonication, and lateral force. In some embodiments, susceptibility to means of removal may be enhanced by incorporation of moieties particularly responsive to means of removal, such as charged or dense moieties for electrophoretic or sedimentation-based removal.

### Location of Analysis

Various locations may be used for sample analysis. In some embodiments, the location of the steps of the analysis, which may be used singly or in combination, include microtiter plates, tubes, the surfaces of particles or beads, nanowell arrays, flow injection analysis apparatus, microfluidic chips, conductive surfaces, temperature-controlled environments, pressure chambers, ovens, irradiation chambers, electrophoretic, field-flow and chromatographic apparatus, microscope stages, luminometers, Coulter principle devices, cantilever and FET sensors, vacuum chambers, electron optical apparatus, single-molecule detection apparatus, single-molecule fluorescence detection apparatus, surfaces bearing nanoholes, electrodes or pillars, emulsions, lateral flow membranes, flow through membranes, lateral flow assay readers, flow through assay readers, gel documentation systems, robotic apparatus, and combinations thereof. Rotation and flow devices, or fast electronic or mechanical shutters, enhance sensitivity by allowing detection of luminescence before it reduces with time. Flow injection analysis, “Lab on a chip” and “Lab on a CD” approaches can be desirable in some embodiments. In some embodiments, it may be advantageous to perform more than one type of analysis in series, either fractionating a sample using or based on the results of one method before performing an additional method, or by interpreting together the results of multiple methods.

In some embodiments, assays may be used to detect specific sequences of nucleic acids. Nucleic acids may be detected from a variety of sample types depending on the application, such as tumor cell lysates for cancer diagnostics. The analyte nucleic acid is recognized by another nucleic acid that functions as MRE. The MRE nucleic acid is physically associated to a CMA such as ligase or a helicase. Assay components are added such that the ligase or helicase generate templates that are specifically associated to the molecular recognition of the MRE nucleic acid to the analyte nucleic acid. These templates are then subject to an amplification step by polymerase chain reaction (PCR), isothermal PCR, loop-mediated isothermal amplification (LAMP), or recombinase polymerase amplification (RPA) amplification, and this amplified signal is detected by appropriate means. Assay elements may be functionalized with single stranded DNA or RNA that hybridizes with part of a complementary strand from a sample that is specific to an analyte. In some embodiments the target complementary nucleic acid strand has a specific tag which is introduced during amplification or by other means, and allows the tagged strand to be captured by a surface that recognizes the tag. For example, an amplification product may be biotinylated so that it binds to surfaces coated with avidin, or is retained at particular locations in a lateral-flow strip. In other embodiments, the surface is functionalized with a nucleic acid strand that specifically hybridizes with a small segment of the target nucleic acid strand, which is long enough to also allow hybridization with a reporter containing a complementary sequence to a different part of the target nucleic acid strand. Multiplexed lateral flow assays can be readily designed by adding multiple test lines to a strip and using reporters functionalized with different molecular recognition elements to bind specifically at each test line.

### EXAMPLES

### Example 1

### DNA Glycosylase as CMA for Ultra-Sensitive and Quantitative Biomarker Detection

In this example, the analyte or biomarker of interest is first captured by a primary ligand such as antibody, aptamer and nanobody etc., immobilized on the surface of a solid phase, such as magnetic nanoparticles or microplates. DNA glycosylase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of DNA glycosylase conjugated ligand captured by the solid phase is proportional to the amount of biomarker captured by primary ligand. The captured DNA glycosylase can be released as free enzymes by cleaving the cleavable linker. In the presence of dsDNA template containing dUTP or other damaged bases in 3′ extremities, the bases are excised. An AP endonuclease will then cleave the nick in the phosphodiester backbone and polymerase with a 5′-3′ exonuclease activity will fill the gap at the 3′ end to form blunt end dsDNA. This dsDNA not containing modified bases can now be copied by high fidelity polymerase such as Q5, Pfu and Phusion polymerase. The amplified signal can be used to detect and quantify the amount of biomarker present.

### Example 2

### AP Lyase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. AP lyase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of AP lyase conjugated ligand captured by the solid phase is proportional to the amount of biomarker. The captured AP lyase can be released as free enzymes by cleaving the cleavable linker. In the presence of dsDNA template containing an apyrimidinic site, the backbone is cleaved polymerase with a 5′-3′ exonuclease activity will fill the gap at the 3′ end to form blunt end dsDNA. This dsDNA is adequate for the PCR amplification with a high fidelity polymerase such as Q5. The amplified signal can be used to detect and quantify the amount of biomarker present.

### Example 3

### DNA or RNA Polymerase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. A DNA Polymerase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of polymerase conjugated ligand captured by the solid phase is proportional to the amount of biomarker. The captured polymerase can be released as free enzymes by cleaving the cleavable linker. In the presence of a DNA with modified bases blocking the PCR reaction such as dUTP, the DNA glycosylase and the AP lyase present in the medium will produce nick in the dsDNA. A polymerase with 5′-3′ exonuclease will remove the DNA containing the modified bases and fill the 3′ recessed end to generate a blunt-DNA with dNTPs suitable for PCR using high fidelity polymerase. The amplified signal can be used to detect and quantify the amount of biomarker present.

### Example 4

### Exonuclease as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Exonuclease as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of exonuclease conjugated ligand captured by the solid phase is proportional to the amount of biomarker. The captured exonuclease can be released as free enzymes by cleaving the cleavable linker. In the presence of dsDNA template containing dUTP or other damaged bases in 3′ extremities, the bases are excised. A polymerase will then fill in then extend the 3′ end to form blunt-end DNA. The exonuclease can be detected by the generation of ssDNA from a dsDNA template. The newly formed ssDNA is protected by attachment of Single-Stranded Binding proteins. The presence of SSB is then detected. The ssDNA may alternatively comprise an aptamer sequence, the binding of which to a selected location is used as a read-out, or ssDNA may be detected using antibodies which bind ssDNA.

### Example 5

### Endonuclease or Nickase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. The endonuclease or nickase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of endonuclease conjugated ligand captured by the solid phase is proportional to the amount of biomarker. The captured endonuclease can be released as free enzymes by cleaving the cleavable linker. In the presence of dsDNA endonuclease cleaves the DNA generating an end recognizable by an exonuclease. The formed ssDNA is detected by SSB. The ssDNA may alternatively comprise an aptamer sequence, the binding of which to a selected location is used as a read-out, or ssDNA may be detected using antibodies which bind ssDNA. In a further embodiment of this example, the endonuclease can be engineered as a dimer or oligomer with 2 or more monomer units connected through a ligand. This dimerized/oligomerized endonuclease can then be covalently conjugated to a secondary ligand (MRE), such as antibody, aptamer and nanobody etc.

### Example 6

### Helicase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Helicase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of helicase conjugated ligand captured by the solid phase is proportional to the amount of biomarker. The captured helicase can be released as free enzymes by cleaving the cleavable linker. The helicase allows formation of ssDNA by separating the two strands of dsDNA. This exposes 2 ends of ssDNA, which can be degraded by exonuclease specific for ssDNA or be captured by Single-Stranded Binding proteins. The unwinding of dsDNA also can promote the binding of primers for PCR reaction such as isothermal PCR. Any of these amplified signals can be used to detect and quantify the amount of biomarker present.

### Example 7

### Methylase-Antibody Conjugate Preserving a DNA Reporter that is Detected By PCR

An antibody recognizing a biomarker protein is chemically fused to a DNA methylase enzyme, and used as the detector antibody in a sandwich ELISA format. A DNA containing the enzyme methylation recognition sequence is added and is incubated with the Ab-enzyme conjugate. A restriction endonuclease that cleaves the unmodified recognition sequence is added. In the presence of the biomarker, the retained Ab-methylase conjugate modifies the DNA and is protected from cleavage by the endonuclease. The protected DNA is detected by PCR, and a lower Ct is interpreted as evidence of the presence of the biomarker.

### Example 8

### Methylase-Antibody Conjugate Labeling DNA or RNA Reporter for Cleavage that is Detected By Absence of PCR Product

An antibody recognizing a protein is genetically fused to an RNA methylase enzyme, and the conjugate used as a reporter in a sandwich immunoassay. An RNA containing the enzyme methylation recognition sequence is incubated with the Ab-enzyme conjugate, and a restriction enzyme that cleaves the modified recognition sequence is added. In the presence of Ab-methylase conjugate the DNA or RNA is modified and cleaved by the endonuclease. The surviving DNA is detected by PCR, and a higher Ct is interpreted as evidence of the presence of the biomarker. DNA detection may alternatively be by another amplification method such as RPA.

### Example 9

### Kinase-Antibody Conjugate Adding Phosphate Group to 5′ End of a Ligatable DNA Reporter that is Detected By PCR

An antibody recognizing an analyte is chemically or genetically fused to a kinase. A DNA reporter is added, which does not contain a phosphate group on its 5′end. The Ab-kinase conjugate is used as a reporter to bind to a protein target bound on a capture antibody in a microwell. Two oligonucleotides (A and B), both complementary to adjacent sequences on a bridge oligo C are added, and the kinase adds a phosphate group to the 5′end of DNA oligo A, thus allowing subsequent ligation to take place between oligo A and oligo B with 3′OH end in presence of bridge oligo C complementary to A and B. Quantitative RPA detection of the ligated product is used to estimate the concentration of the analyte.

### Example 10

### Kinase-Antibody Conjugate Removing a Phosphate Group from 3′End of DNA or RNA Reporter, Allowing Formation of a Ligated Product Which is Then Detected By PCR

An antibody recognizing an analyte is genetically fused to a kinase. A DNA reporter is added, which contains a phosphate group on the 3′end. In the presence of Ab-kinase conjugate retained by binding to an analyte, the kinase removes the phosphate group from the 3′end of DNA oligo A, thus allowing ligation to take place between oligo A and the 5′ phosphorylated end of oligo B in presence of a bridge oligo C complementary to A and B. Quantitative digital PCR detection of the ligated product is used to estimate the concentration of the analyte.

### Example 11

### Kinase-Antibody Removing Phosphate Group from 3′End and Adding Phosphate Group to 5′End of a DNA or RNA Reporter Which is Then Detected By PCR

An antibody recognizing an analyte is co-immobilized on a phage VLP particle with a kinase. A DNA or RNA reporter is added which contains a phosphate group on 3′end. In the presence of Ab-kinase, the kinase removes the phosphate group from 3′end of DNA or RNA oligo A and adds a phosphate group to 5′end of DNA or RNA B, thus allowing ligation to take place between oligos A and B in presence of a bridge oligo C complementary to A and B. The signal from the reporter is used to detect and quantify the amount of analyte present.

### Example 12

### DNA or RNA Endonuclease-Antibody Conjugate Including RNA-Guided Endonuclease Cleaving DNA or RNA Reporter Which is Then Detected By Absence of PCR Product

An antibody recognizing an analyte is chemically or genetically fused to an endonuclease. The conjugate is retained in a volume by captured analyte. A DNA or RNA reporter is added, which contains the enzyme's recognition site. In the presence of Ab-endonuclease conjugate, the DNA or RNA is cleaved. The signal from the reporter is used to detect and quantify the amount of analyte present.

### Example 13

### Phosphatase-Antibody Conjugate Enabling Ligation to Take Place

An antibody recognizing an analyte is chemically or genetically fused to a kinase. A DNA or RNA reporter is added, which contains a phosphate group on 3′end. In the presence of Ab-kinase, the kinase removes phosphate group from 3′end of DNA or RNA oligo A, allowing ligation to take place between oligo A and 5′ phosphorylated oligo B in presence of a bridge oligo C complementary to oligos A and B. The signal from the reporter is used to detect and quantified the amount of analyte present.

### Example 14

### Phosphatase-Antibody Conjugate Labeling DNA or RNA Reporter for Degradation Which is Then Detected by Absence of PCR Product

An antibody recognizing an analyte is chemically or genetically fused to a phosphatase. A DNA or RNA reporter is added which contains a phosphate group on 3′end. In the presence of Ab-phosphatase, the phosphatase removes the phosphate group from 3′ end leaving DNA or RNA degradable by an exonuclease. The signal from the reporter is used to detect and quantified the amount of analyte present.

### Example 15

### Using DNA Ligase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. DNA ligase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of DNA ligase conjugated ligand captured by the solid phase is proportional to the amount of biomarker. The captured DNA ligase optionally can be released as free enzymes by cleaving the cleavable linker. The released free enzymes are introduced into a system containing at least single strand DNA: oligos A, B and C, and optimized buffer conditions. Only with the enzyme, the A and B oligos will be ligated and further be sensitively and quantitatively detected by competitive PCR.

### Example 16

### Using Uracil-DNA Glycosylase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Uracil-DNA Glycosylase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of Uracil-DNA Glycosylase conjugated ligand captured by the solid phase is proportional to the amount of biomarker. The captured Uracil-DNA Glycosylase can be released as free enzymes by cleaving the cleavable linker. The released free enzymes are introduced into a system containing at least one double strand DNA: oligo A containing deoxyuracil and optimized buffer conditions. Only with the enzyme, the deoxyuracil in A will be removed and further be recognized by the designed primers and thereby sensitively and quantitatively detected by competitive PCR.

### Example 17

### Using Ubiquitin Protein Ligase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Ubiquitin Protein Ligase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of Ubiquitin Protein Ligase conjugated ligand captured by the solid phase is proportional to the amount of biomarker. The captured Ubiquitin Protein Ligase can be released as free enzymes by cleaving the cleavable linker. The released free enzymes are introduced into a system containing at least ubiquitin, highly detectable reagents (such as Horseradish peroxidase (HRP) or phage) carrying ubiquitin modification sites and optimized buffer conditions. Only with the enzyme, the highly detectable reagents will be conjugated with ubiquitin and captured by another solid phase whose surface is modified with ubiquitin binding ligands and thereby be sensitively and quantitatively detected.

### Example 18

### Using Cre Recombinase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Cre Recombinase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of Cre Recombinase conjugated ligand captured by the solid phase is proportional to the amount of the biomarker. The captured Cre Recombinase can be released as free enzymes by cleaving the cleavable linker. The released free enzymes are introduced into a system containing at least one double strand DNA: A containing at least two LoxP sites and optimized buffer conditions. Only with the enzyme, a circular double strand DNA: B will be generated from A and thereby sensitively and quantitatively detected by competitive PCR.

### Example 19

### Using Lipoic Acid Ligase as CMA for Ultra-Sensitive and Quantitative Biomarker Detection

In this example, the analyte or biomarker of interest is first captured by a primary ligand, such as antibody, aptamer and nanobody etc., immobilized on the surface of a solid phase, such as magnetic nanoparticles or microplates. Lipoic Acid Ligase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of Lipoic Acid Ligase conjugated ligand captured by the solid phase is proportional to the amount of biomarker. The captured Lipoic Acid Ligase optionally can be released as free enzymes by cleaving the cleavable linker. The released free enzymes are introduced into a system containing at least a p-iodophenyl derivative, highly detectable reagents (such as Horseradish peroxidase (HRP) or phage) carrying 13-amino-acid lipoic acid acceptor peptide sequence (LAP) and optimized buffer conditions. Only with the enzyme, the highly detectable reagents will be conjugated with p-iodophenyl derivative and subsequently conjugated to another solid phase through palladium mediated Sonogashira cross-coupling and thereby be sensitively and quantitatively detected.

### Example 20

### Streptavidin or Streptavidin Modified Nanoparticles as CMA for Ultra-Sensitive and Quantitative Biomarker Detection

In this example, the analyte or biomarker of interest was first captured by a primary ligand, such as antibody, aptamer and nanobody etc., immobilized on the surface of a solid phase, such as magnetic nanoparticles or microplates. Streptavidin (or modified Steptavidin such as ExtrAvidin or NeutrAvidin) or streptavidin coated nanoparticles as the CMAs are covalently conjugated through a cleavable linker to a secondary ligand (MRE), such as antibody, aptamer and nanobody etc., which specifically binds to the biomarker of interest without interfering the binding between the biomarker and the primary ligand. The amount of conjugated ligand captured by the solid phase was proportional to the amount of biomarker. The captured streptavidin (or modified Steptavidin such NeutrAvidin) or streptavidin coated nanoparticles was released as free reporters by cleaving the cleavable linker. These streptavidin (or modified Steptavidin such NeutrAvidin) or streptavidin coated nanoparticles were introduced into a system containing at least 5′-biotinylated single-strand DNA oligo-A, 3′-biotinylated-5′-phosphate single-strand DNA oligo-B, single-strand DNA oligo-C containing deoxy uracil, which can both hybridize with oligo-A and oligo B, and optimized buffer conditions. Only with streptavidin (or modified Steptavidin such NeutrAvidin) or streptavidin coated nanoparticles, the oligos A and B will be ligated and further be sensitively and quantitatively detected by competitive PCR.

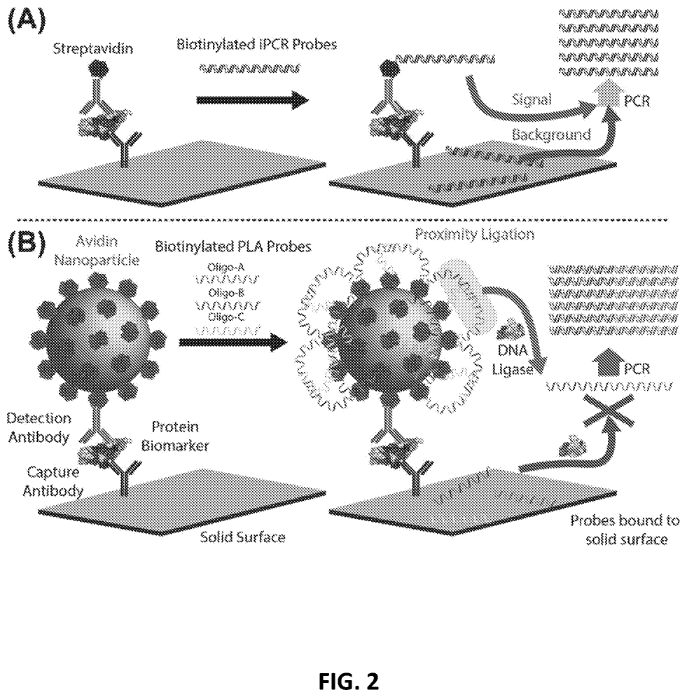

As shown in FIG. 2(A), in traditional Immuno-PCR (iPCR), the PCR template oligo is directly used as the reporter. Non-specifically bound oligos are PCR-amplifiable giving rise to an increased nonspecific background signal. As shown in FIG. 2(B), in nanoparticle-based proximity ligation assay (NP-PLA), avidin-coated nanoparticles serve as the reporters. The avidin nanoparticles bring the two split parts (biotinylated oligo-A and biotinylated oligo-B) of the PCR template and the biotinylated bridge oligo-C into proximity. Then oligo-A and oligo-B are ligated by DNA ligase and the resulting oligomer serves as the PCR template. Any non-specifically bound oligos in NP-PLA cannot be ligated into a PCR-amplifiable template and thus the non-specific background signal is significantly decreased.

Different types of avidin/streptavidin coated nanoparticles were diluted to varied concentrations in 2% BSA, PBS pH 7.4. To each PCR tube, 2 μL of nanoparticle dilution, 1 μL of 60 pM mixture of Oligo-A and Oligo-B and 1 μL of 60 pM of Oligo-C were added, mixed and incubated for 30 min at 25° C. Thereafter, 100 μL of ligation mix (10 μL of 10× buffer for T4 DNA ligase with 10 mM ATP, 24 units of T4 DNA ligase and 90 μL of pure water) was added to each of the PCR tubes, mixed and incubated for 15 min at 25° C. Immediately after the ligation, 10 μL of each reaction solution was mixed with 10 μL of 2× QPCR Master Mix (containing 1 μM of the primers) in another optical PCR tube, and then amplified by PCR (95° C. for 10 min, then 50 cycles of 95° C. for 15 s and 60° C. for 30 s) in an Agilent Mx3005P QPCR System. The −delta Cₜ values are calculated by subtracting the Cₜ value of samples from the Cₜ value of the blank (no particles) control.

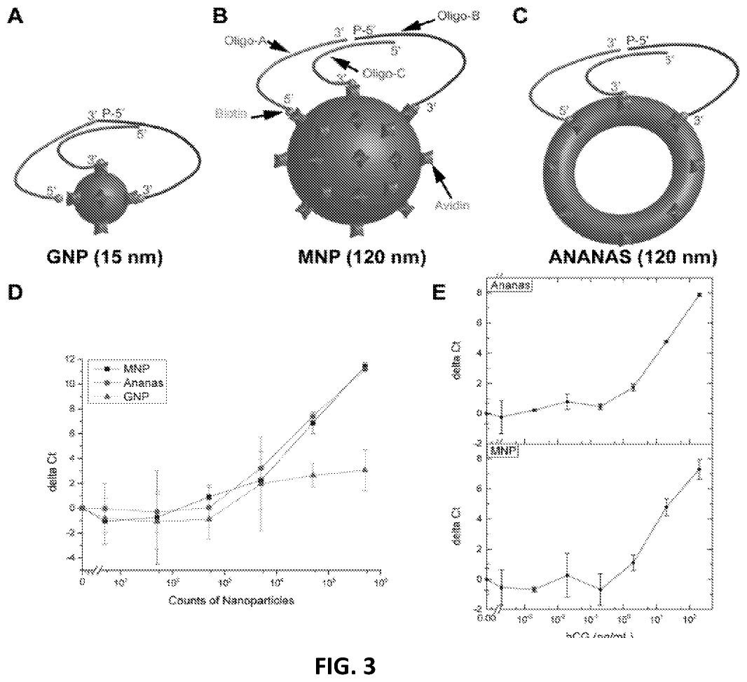

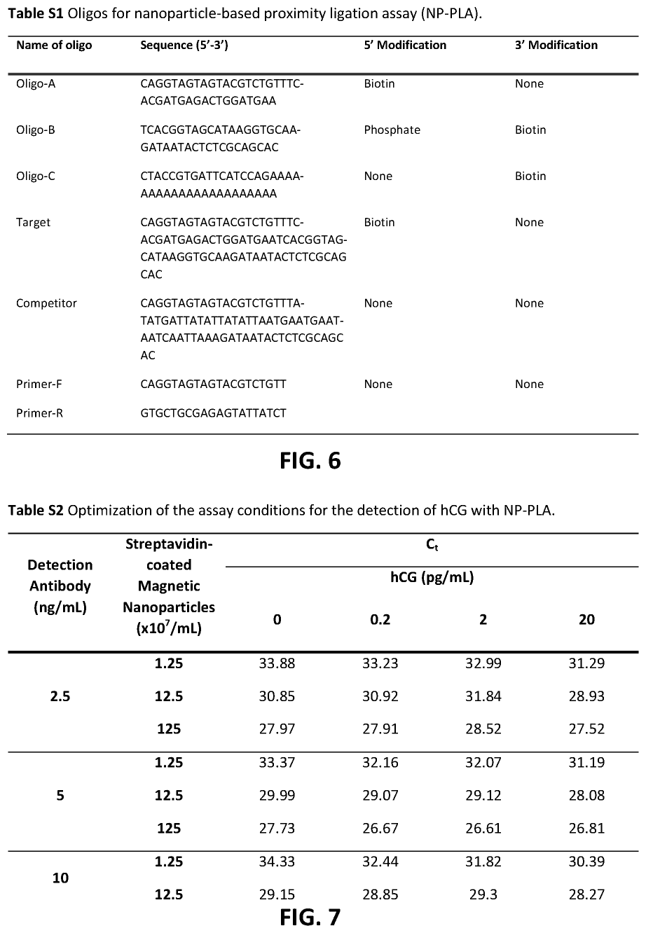

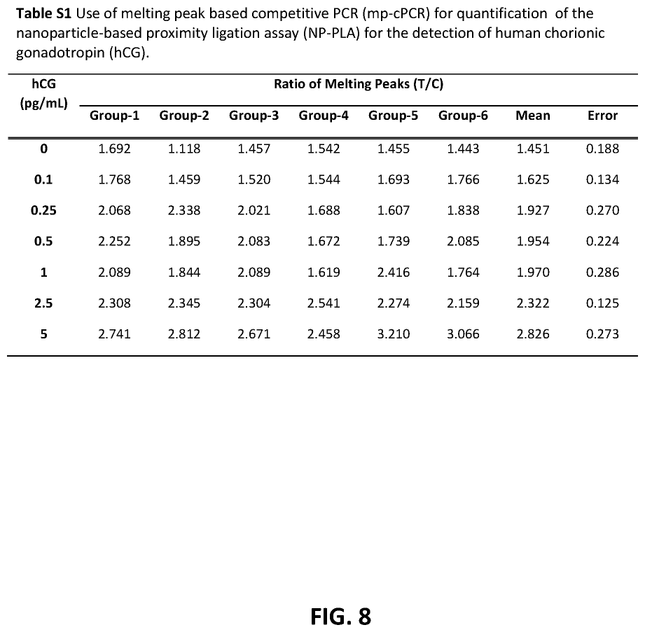

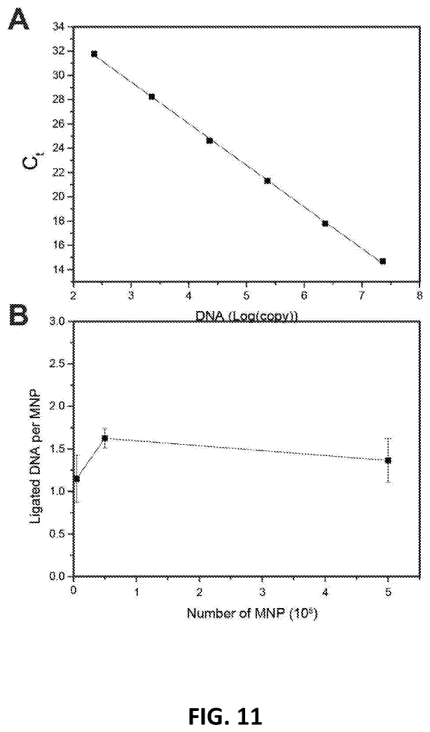

To test the feasibility of detecting avidin nanoparticles with PLA, three commercially available avidin/streptavidin coated nanoparticles (streptavidin coated spherical gold nanoparticles (EM.STP15, BBI Solutions) with mean diameter 15 nm, streptavidin-coated magnetic nanoparticles (Bio-Adembeads Streptavidin plus 0321) with mean diameter 120 nm, and ANANAS nanoparticles (ANANAS nanotech) with mean DLS-effective diameter 120 nm, as shown in FIG. 3, were tested with PLA using a set of optimized oligos as shown in FIGS. 6 and 7. All three types of avidin/streptavidin-coated nanoparticles decreased the Cₜ values of the PLA products in a dose-responsive manner. While both the 120 nm magnetic nanoparticles and 120 nm ANANAS nanoparticles showed similar limit of detection (LOD) of around 500 particles and similar slope of delta Cₜ as a function of number of nanoparticles, the 15 nm gold nanoparticles showed a 10-fold higher LOD and flatter −delta Cₜ/nanoparticle concentration slope, indicating that PLA efficiency is higher with avidin/streptavidin-coated nanoparticles of bigger size compared to those of smaller size. As ANANAS nanoparticles showed highest detectability with PLA, they were selected for the following development of the NP-PLA. The ligation yield from avidin-coated nanoparticles was also analyzed, as shown in FIG. 11. Based on a real-time quantitative PCR (qPCR) standard curve of full length ligation product, the ligation yield is a constant of ˜1.5 per the 120-nm avidin particle on average with current settings. Further optimization of the NP-PLA system may increase the ligation yield.

### Example 21

### Preparation and Characterization of DNA Avidin Nanoparticles

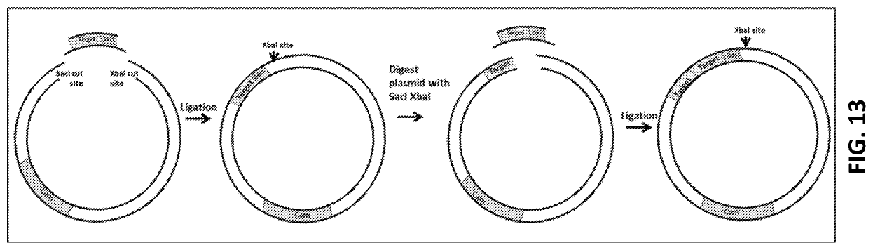

A plasmid containing several copies of a specific template (80 bp) was constructed using pBC SK plasmid. pBC plasmid was linearized using SacI and XbaI as shown in FIG. 13. The target sequence (80 bp) ordered had 5′ and 3′ ends compatible with SacI and XbaI sites respectively. In addition, downstream of target sequence at 3′ end, a SacI site was introduced in the synthesized oligo. Once the oligo was ligated to linearized pBC, the SacI site in pBC was eliminated. To introduce the second or additional copies of target sequence, the plasmid with one copy of template was linearized with SacI and XbaI enzymes and ligated to the synthesized double stranded oligo. Plasmids were transformed into recA mutant cells to ensure stability of constructs. Plasmids containing up to 7 copies of template (target sequence) was synthesized using similar protocol. The size of plasmid with 1 copy of template was 3500 bp. In the successive plasmids when additional copies template were introduced, the size of plasmid increased by 85 bp for every copy of template introduced. DNA copy number of plasmid (containing respective copies of template) was determined using concentration obtained from Nanodrop, which was then multiplied to Avogadro's number to give the final value of plasmid DNA copies/ml. For the construction of DNA-Avidin nanoparticles, in an eppendorf tube, the plasmid DNA (containing desired copies of template) was diluted using deionized water to obtain the final concentration of 1×10¹² DNA copies/ml. Avidin (Life Technologies) was pre-diluted in a separate eppendorf tube using deionized water to a concentration such that when DNA was added to the Avidin solution, there was one Avidin molecule for every 4 bp. The concentration of Avidin was dependent on the length of plasmid containing the desired template copies. Two eppendorf tubes containing plasmid DNA and Avidin were placed on ice for 15-30 mins. After incubation, the plasmid DNA was added to the Avidin tube such that final volume was 1 ml. The tube was immediately vortexed for 30 sec. DNA and Avidin were allowed to interact for 1 h at room temperature on a rotator.

A 2 arm poly(ethylene glycol)-biotin (PEG-Biotin, 10 k, Nanocs) was diluted using deionized water such that PEG-Biotin occupied 30% of Avidin binding sites when it was added to DNA-Avidin mixture. After 1 h of incubation, PEG-Biotin was added to the DNA-Avidin tube and the reaction was allowed to take place for 24 h at 4° C. on a rotator. The mixture containing DNA-Avdin nanoparticles was then filtered using Amicon Ultra-0.5, 100 kDa membrane filters (Sigma Aldrich) to remove free Avidin and PEG-Biotin molecules. The particles were then wash with water four times. Purified DNA-Avidin nanoparticles obtained were then used for further analysis. DNA-Avidin nanoparticles can be alternately labeled with Atto-520 biotin to observe under fluorescent microscope.

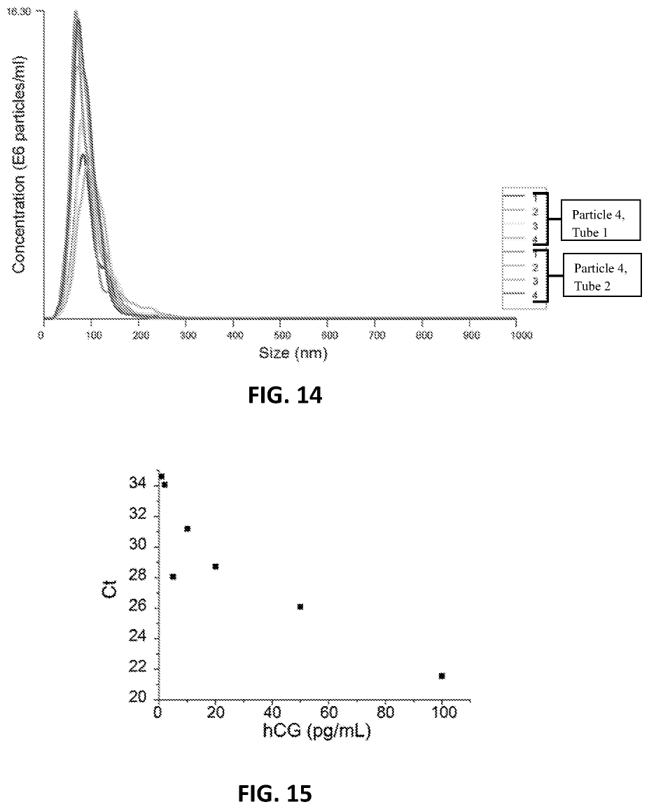

As shown in FIG. 14, the approximate concentration of particles was found to be 7.22×10¹⁰ particles/ml. The sample was diluted 100 times to analyze in NanoSight. Four different fields of views for each sample tube were observed to confirm nanoparticles were uniformly dispersed in solution. The zeta potential of these nanoparticles was found to be 1.17±1.4 mV in contrast to plasmid DNA (−5.2±2.4 mV).

### Example 22

### Use of DNA Template in ANANAS Nanoparticles Amplified Using RPA

Next, an experiment was conducted to determine whether DNA template in ANANAS nanoparticles could be amplified using RPA.

ANANAS Poly-Avidin nanoparticles were obtained from ANANAS nanotech, S.r.l. (Padova, Italy). A stock solution of ANANAS nanoparticle (concentration 1.25×10¹² nanoparticles/ml) was serially diluted using deionized water in a sterile PCR tube to obtain the concentration of 5×10¹⁰ nanoparticles/ml. The 5×10¹⁰ nanoparticles/ml containing PCR tube was then subjected to heat at 95° C. for 10 mins. The heat treated ANANAS nanoparticles were then added to RPA reaction pellets to obtain the concentration 5×10⁸, 5×10⁷ and 5×10⁶ nanoparticles/reaction.

Recombinase polymerase amplification (RPA) was performed using the TwistAmp® Basic kit (TwistDx). Four RPA reaction pellets were rehydrated with 29.5 μl TwistDx rehydration buffer each. To these rehydrated pellets, 0.24 μl of 100 μM forward primer and 100 μM reverse primer specific for DNA template in ANANAS nanoparticle were added in each reaction tube. 1 μl of SYBR green dye (10× working stock) along with 10 μl template (different concentrations of nanoparticles) were added. In no-template control (NTC), 10 μl deionized water was added instead of ANANAS nanoparticles. Deionized water was added in all four reaction tubes to make up the volume to 47.5 μl before the addition of magnesium acetate. RPA reaction was then initiated by addition of 2.5 μl of 280 mM magnesium acetate. RPA reaction tubes containing ANANAS template were then amplified (42° C. for 30 s 60 cycles) with an Agilent Mx3005P QPCR System.

As shown in FIG. 17, amplification was observed in the RPA reaction pellets containing 5×10⁸, 5×10⁷ and 5×10⁶ ANANAS nanoparticles/reaction at 4.24, 4.82 and 4.88 mins respectively. The no-template control showed delayed amplification at 9.5 mins.

It was determined that heating of the ANANAS nanoparticle is useful to make the DNA template accessible for primers to bind and amplify. Using this concept, ANANAS nanoparticles modified with a molecular recognition agent such as an antibody can be used as a highly detectable label for ultra-sensitive human chorionic gonadotropin (hCG) detection.

### Example 23

### Streptavidin Coated Magnetic Nanoparticles as CMA for Ultra-Sensitive Human Chorionic Gonadotropin (hCG) Detection

The feasibility of NP-PLA for detecting human chorionic gonadotropin (hCG) was further investigated. Bovine serum albumin (BSA, A7906-50G), streptavidin-horseradish peroxidase (HRP) conjugate (S5512-0.5 mg), human Chorionic Gonadotropin (hCG, CG10-1VL, using the conversion factor 9.28 IU/ug from the 3rd International Standard), TWEEN® 20 (Molecular Biology Grade, P9416-100 ML), and Nunc® MicroWell™ 96 well polystyrene plates (P7366-1CS) were obtained from Sigma-Aldrich, Inc. (St. Louis, Mo., USA). Pierce premium grade Sulfo-NHS-SS-Biotin (PG82077) and Zeba™ spin desalting columns (89882) and 1-Step™ Ultra TMB-ELISA Substrate Solution (34028) were obtained from Thermo Fisher Scientific, Inc. (Rockford, Ill., USA). Polymerase chain reaction (PCR) optical tubes and caps (8× strips), Brilliant III Ultra-Fast SYBR® Green QPCR master mix, and Agilent Mx3005P QPCR System were obtained from Agilent Technologies, Inc. (Santa Clara, Calif., USA). Phosphate Buffered Saline (PBS) tablets (T9181), pH 7.4 were obtained from Clontech Laboratories, Inc. (Mountain View, Calif., USA). Mouse monoclonal anti-β hCG antibody (ABBCG-0402), Goat anti-α hCG polyclonal antibody (ABACG-0500), and Goat anti-Mouse polyclonal antibody (ABGAM-0500) were obtained from Arista Biologicals, Inc. (Allentown, Pa., USA). T4 DNA Ligase (M0202L), 10× buffer for T4 DNA ligase with 10 mM ATP (B0202S), were obtained from New England Biolabs, Inc. (Ipswich, Mass., USA). (D,L)-1,4-Dithiothreitol (DTT), 99.5+%, Molecular Biology Grade CAS #[27565-41-9] was obtained from Soltec Ventures, Inc. (Beverly, Mass., USA). EM.STP15-15 nm gold streptavidin particles were obtained from BBI DETECTION, INC. (Madison, Wis., USA). Bio-Adembeads Streptavidin plus 0321 was obtained from Ademtech, SA. (Pessac, France). ANANAS Poly-Avidin nanoparticles were obtained from ANANAS nanotech, S.r.l. (Padova, Italy). Infinite® M200 PRO multimode reader and the HydroFlex microplate washer were obtained from Tecan, Co. (Männedorf, Switzerland). Anonymized serum samples were obtained from the Gulf Coast Regional Blood Center (Houston, Tex., USA). All DNA oligos were obtained from Integrated DNA Technologies, Inc. (Coralville, Iowa, USA).

The biotinylation of antibodies used Pierce premium grade Sulfo-NHS-SS-Biotin, following the manufacturer's protocol. The protein was mixed with sulfo-NHS-SS-Biotin (mole ratio, 1:20), and reacted at room temperature for 30 min. The uncoupled sulfo-NHS-SS-Biotin was removed with a Zeba™ desalting column (40 KDa MW) according to the manufacturer's protocol. After biotinylation, the mole ratio of biotin to antibodies was estimated to be 4˜4.5 using the 4′-hydroxyazobenzene-2-carboxylic acid (HABA) assay. Samples of biotinylated proteins were mixed with HABA/avidin reagent for at least 2 min at 25° C. The changes of absorbance at 500 nm were recorded and used to calculate the amount of biotin in the samples of biotinylated proteins. The biotinylated antibodies were stored with 1% BSA in PBS pH 7.4 at 4° C.

Enzyme-linked immunosorbent assay (ELISA). Selected wells of a Nunc® MicroWell™ 96 well polystyrene plate were each coated with 100 μl capture antibody (10 μg/mL mouse monoclonal anti-β hCG antibody (Arista Biologicals Inc. ABBCG-0402) for hCG detection) in PBS pH 7.4, overnight, at 4° C. Thereafter, the antibody solutions were removed from these wells and 300 μL PBS with 3% BSA was added to each well, for 2 h, at 25° C. Then these wells were washed 3 times with PBS, 0.1% TWEEN® 20. Samples (100 μL) were immediately added to each of the wells after washing and incubated for 1.5 h at 25° C. Then these wells were washed 3 times with PBS, 0.1% TWEEN® 20. Buffer A (PBS pH 7.4 with 1% BSA) was used to make dilutions in the following steps. Detection antibodies (100 μL, 10 ng/mL of biotinylated goat anti-α hCG polyclonal antibody (ABACG-0500) for hCG detection) were then added to each of the wells and incubated for 30 min at 25° C. Then these wells were washed 3 times with PBS, 0.1% TWEEN® 20 and streptavidin-HRP added to each well and incubated overnight, at 4° C. Wells were then washed 3 times with PBS, 0.1% TWEEN® 20 and 100 μL of 1-Step™ Ultra TMB-ELISA Substrate Solution was added to each well and incubated for 20 min at 25° C. Finally, 50 μL of 2 M sulfuric acid was added to each well and the absorbances measured and recorded with an Infinite® M200 PRO multimode reader.

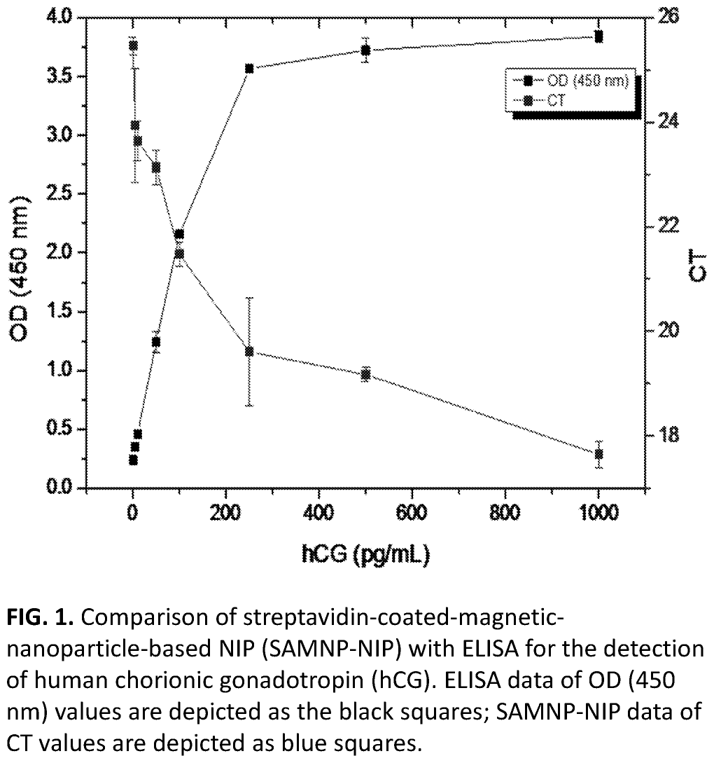

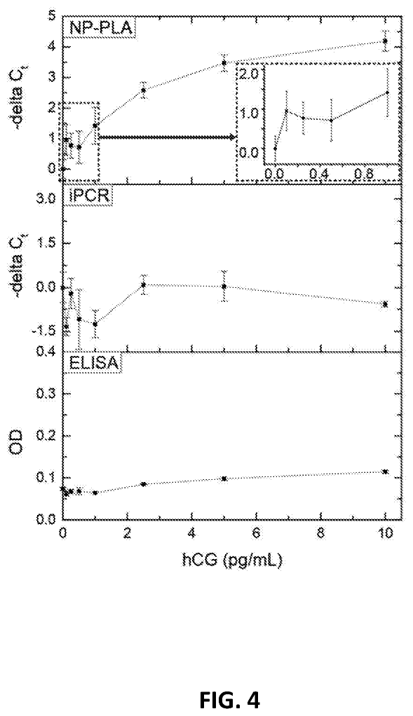

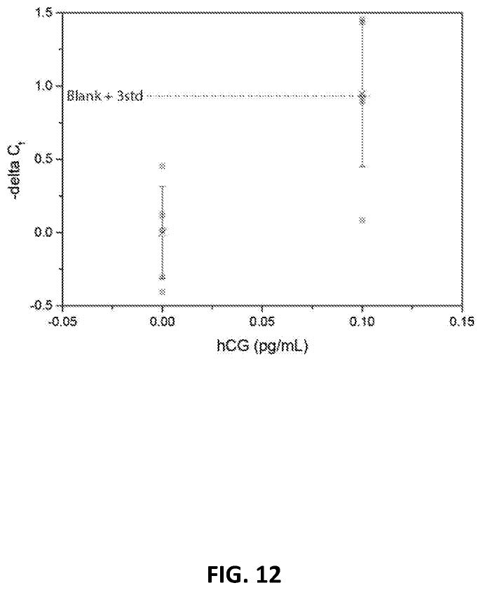

In the hCG detection scheme, monoclonal antibodies recognizing the β-subunit of hCG were immobilized on the surface of the wells of a microplate; biotinylated polyclonal antibodies recognizing the α-subunit of hCG were tagged with ANANAS nanoparticles by biotin-streptavidin linkage with a disulfide bond; the −delta Cₜ increased logarithmically with the amount of hCG, with an LOD of 100 fg/mL (FIG. 12; 2.6 fM, 100 μL sample volume) demonstrating the feasibility of the NP-PLA. Compared to iPCR and enzyme-linked immunosorbent assay (ELISA) with same assay settings, NP-PLA is 100 times more sensitive (see FIGS. 1 and 4).

Nanoparticle-based Proximity Ligation Assay. The first part of the NP-PLA protocol is the same as that for the ELISA. After the incubation of the detection antibodies, the wells were washed 3 times with PBS, 0.1% TWEEN® 20. All the following dilutions, except as noted, were made in Buffer A (PBS pH 7.4 with 1% BSA). Thereafter, 100 μL of avidin-coated nanoparticles (1.25×10⁷/mL) were added to each of the wells and incubated overnight, at 4° C. Then these wells were washed 3 times with PBS, 0.1% TWEEN® 20. Thereafter, 50 μL of the mixture of 60 pM Oligo-A and Oligo-B and 50 μL of 60 pM Oligo-C was added to each well and incubated for 30 min at 25° C. The wells were washed 3 times with PBS, 0.1% TWEEN® 20 and 30 μL 50 mM DTT in water was added to each well and incubated for 2 h at 25° C. Finally, 70 μL of ligation mix (10 μL of 10× buffer for T4 DNA ligase with 10 mM ATP, 24 units of T4 DNA ligase and 60 μL water) were added to each well, mixed and incubated for 15 min at 25° C. Immediately after ligation, 10 μL of each reaction solution was mixed with 10 μL of 2× QPCR Master Mix (containing 1 μM of the primers) in another optical PCR tube, and then amplified by PCR (95° C. for 10 min 1 cycle, then 50 cycles of 95° C. for 15 s and 60° C. for 30 s) with an Agilent Mx3005P QPCR System. The −delta Cₜ values are calculated by subtracting the Cₜ value of samples from the Cₜ value of the blank (no target analyte) control.

To precisely quantify protein biomarkers at ultra-low levels, melting peak based competitive PCR (mp-cPCR) was used to quantify the NP-PLA results. In the mp-cPCR scheme, a competitor sequence was designed (shown in FIG. 6) with the same primer binding sites but with a melting temperature 10° C. lower than the target sequence of the ligation products. Ligation products were coamplified with 30 copies of the competitor sequence added as an internal standard in the same PCR tubes.

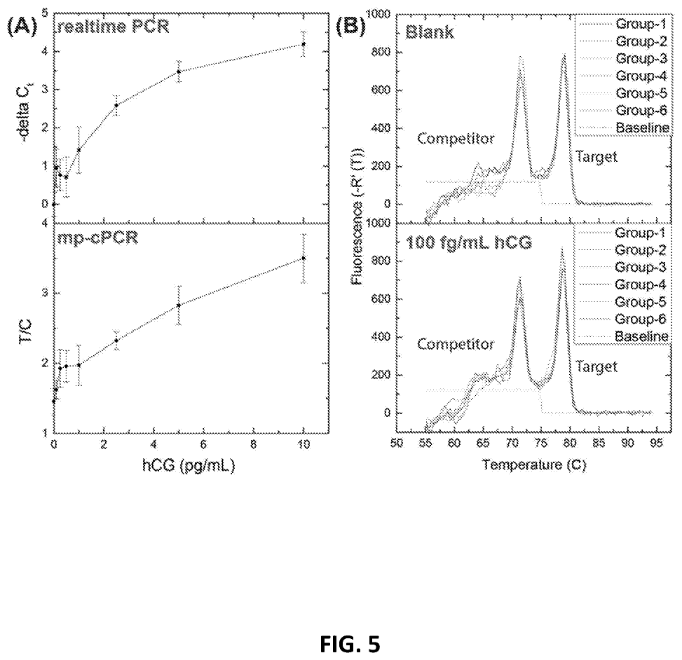

As shown in FIG. 5A, instead of a logarithmic increase, the ratios between the peak-areas of target sequence and competitor sequence (T/C) in mp-cPCR increased linearly with the concentration of hCG in the NP-PLA for hCG detection, showing the utility of mp-cPCR for quantification in NP-PLA. As shown in FIG. 5B, with mp-cPCR the LOD remained at 100 fg/mL of hCG (2.6 fM, 100 μL sample volume), at which hCG level the ratio between the peak areas of target and competitor (1.62±0.13) is significantly higher than the blank controls (1.45±0.19) in all sextuplicates, demonstrating the feasibility of mp-cPCR for NP-PLA.

### Example 24

### Using Competitive PCR for Quantitative Detection of Protein with Streptavidin Coated Magnetic Nanoparticles as CMA

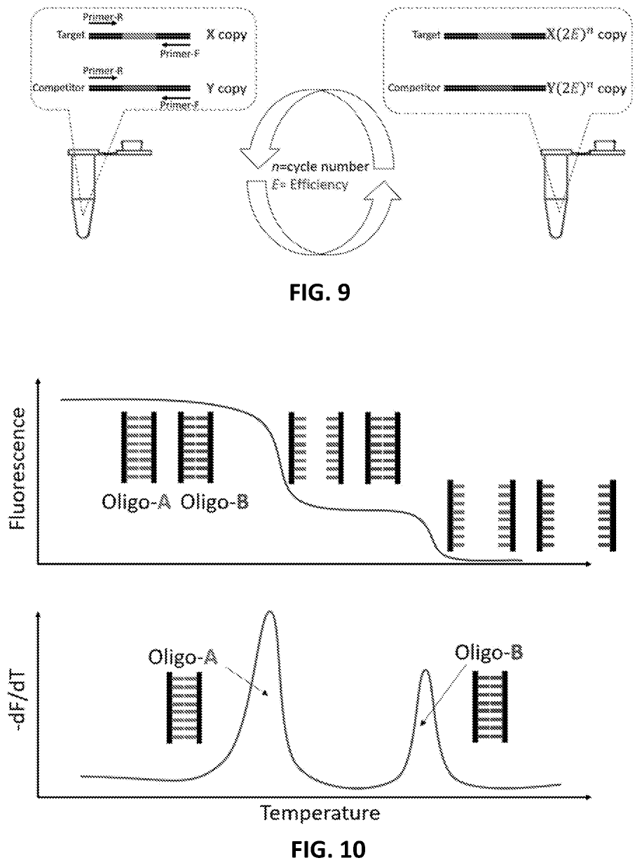

In this example, the real time PCR result are analyzed in a competitive way. An internal competitor DNA oligo set using the same primers but without biotinylation and producing amplicon with a different melting temperature are premixed in the oligo-DNA ligase mix, as shown in FIG. 9. The amounts of target protein to be detected are indicated by the ratios between the peak areas of the two amplicons in the melting curve, as shown in FIG. 10. To assess the melting curve based competitive PCR, 30 copies of the competitor oligo were added as an internal standard to the ligation products of each PCR reaction. Control reactions with 30 copies of the target oligo and/or 30 copies of the competitor oligo were used to determine the positions and the baselines of the target and competitor peaks in the melting curve. One cycle of 95° C. for 60 s, 55° C. for 30 s and 95° C. for 60 s was added at the end of the PCR amplification. The fluorescence was continuously recorded while heating from 55° C. to 95° C. to perform the melting curve analysis. The melting curves are analyzed with OriginPro 9.0.

By using the competitive way, the amount of target proteins at lower range can be quantified with higher resolution.

### Example 25

### ANANAS Poly-Avidin Nanoparticles as CMA for Ultra-Sensitive Human Chorionic Gonadotropin (hCG) Detection

In this example, ANANAS Poly-Avidin nanoparticles are used as the reporter for human chorionic gonadotropin (hCG) detection. On the bottom of 96-well microplate wells, anti-β hCG monoclonal antibody are coated. Samples are incubated in the coated wells for an hour. The wells are then washed. Anti-α hCG polyclonal antibody attached with DTT-cleavable linkers to ANANAS-like particles comprising ExtrAvidin, a plasmid containing 3 copies of an amplifiable DNA sequence, and poly(ethylene glycol) are incubated with the wells. The wells are then washed. ANANAS-like nanoparticles are thereby incubated with the wells. The wells are treated with dithiothreitol (DTT). An aliquot of this DTT treated reaction samples are heated at 95° C. for 10 mins and are then added as template to RPA reaction mix for amplification.

### Example 26

### Using Aptamers on Nanoparticles Also Bearing a Modifying Enzyme on a Cleavable Linker

A biotinylated aptamer recognizing Norwalk virus is covalently coupled to streptavidin magnetic nanoparticles, which also are modified with modifying enzyme by a cleavable linker. After binding to a surface decorated with anti-Norwalk antibodies and treated with samples potentially containing Norwalk virus, a magnetic field is applied to remove non-specifically bound particles, cleave the linker to liberate the modifying enzyme, add DNA substrate of the modifying enzyme, then amplify by PCR those molecules preserved by the modifying enzyme.

### Example 27

### BirA-Mediated Detection of a Protein Analyte

BirA is an E. coli biotin ligase that specifically conjugates biotin to Avitag, a 15-aa sequence (GLNDIFEAQKIEWHE). In this embodiment, capturing magnetic particles functionalized with antibodies or antibodies adsorbed on a microwell plate are used to capture the protein analyte from the samples. Detection antibodies tagged with the Avitag peptide are then offered to bind. After washing and concentration, BirA enzyme is offered to specifically biotinylate the Avitag peptide on the detection antibodies. A mixture of streptavidin gold nanoparticles and biotinylated DNA reporters is then offered at a specific ratio to ensure available streptavidin binding sites. After incubation and washing of non-bound biotinylated DNA reporters, sensitive detection of bound DNA reporters and thus detection of the captured protein analyte is done by PCR or isothermal RPA.

### Example 28

### Wash-Free DNA Detection Assay

In this embodiment, streptavidin agarose (a low non-specific binding scaffold) particles modified with desthiobiotin-labeled hairpin molecular beacons are used to detect a DNA biomarker that hybridizes to the single-stranded loop of the molecular beacon and thus becomes protected from nucleases. Unbound reporters are degraded by Aspergillus nuclease S1 (an endonuclease enzyme derived from Aspergillus oryzae that degrades single-stranded DNA (ssDNA) into mononucleotides. In the presence of excess biotin, the released dsDNA fragment can be quantitatively detected by PCR or isothermal RPA.

### Example 29

### LFA Detection of Protein Biomarkers

In this embodiment, capturing magnetic particles functionalized with antibodies or antibodies adsorbed on a microwell plate are used to capture the protein analyte from the samples. Detection antibodies tagged with nanogold-labeled ssDNA reporters via a cleavable linker (e.g. desthiobiotin, SPDP) are then offered to bind to the captured protein analyte. After washing to remove non-specific bound molecules, the DNA reporters are released and detected in an LFA strip bearing complimentary DNA test lines. This embodiment is easily multiplexable when comprised of different detection antibodies tagged with different ssDNA reporters. The released DNA reporters are detected in an LFA strip bearing multiple test lines.

### Example 30

### Detection of NS1 Protein

Two monoclonal antibodies recognizing different but adjacent epitopes on DENV NS1 protein are covalently coupled to two synthetic DNA probes, DNA1 and DNA2 that are amine-modified at its 5′-end and 3′-end, respectively. The amine-modified DNA probes are conjugated to the periodate-oxidized glycosylated residues on the Fc of the antibodies. The orientated binding of the antibodies on NS1 protein in solution brings the DNA probes into close proximity and in the presence of a connector oligonucleotide fragment that hybridizes to DNA probes and DNA ligase, the gap is ligated and the resulting DNA strand can be amplified by real-time PCR or isothermal RPA.

### Example 31

### Elastase-Mediated Protein Detection

In this embodiment, capturing antibodies adsorbed on a microwell plate or on magnetic beads are used to capture the protein analyte from the samples. Detection antibodies tagged in a 1:1 ratio with human neutrophil elastase are then offered to bind. After washing of non-specific binding molecules, the bound enzyme is quantitative detected using a fluorogenic or chromogenic peptide elastase substrate.

### Example 32

### Ultra-Sensitive Detection of Protein and Non-Protein Analytes Using Antibody Restriction Enzyme Conjugate as the Reporter

In one example, the technology introduced here can be used for the ultrasensitive detection of protein and non-protein analytes. Target analytes are captured by specific polyclonal antibodies immobilized on a universal, solid surface with low non-specific binding and then recognized by the antibody conjugated to restriction enzyme. After extensive washing for enhanced specificity, reaction mixture containing three DNA oligonucleotides (A, B, and C), ATP, divalent cations, ligase, and other ligation buffer components. Oligonucleotide A is a double stranded DNA or partially double stranded DNA at its 3′ end. Its 3′ terminal contains a specific sequence that is the reverse complement to 3′ half of the ligation bridge oligonucleotide C, and is flanked by a restriction enzyme recognition sequence. Oligonucleotide B contains a specific sequence on its 5′ terminal which complements the other half of the ligation bridge oligonucleotide C on 5′ terminal. The presence of endonuclease restriction enzyme, which recognizes the cutting site on oligonucleotide A and cuts off the 3′ end, allows ligation between oligonucleotides A and B held together by C. PCR is performed using primer set that span across A and B oligonucleotides to amplify the ligated product.

In a modification, oligonucleotide A is a single stranded DNA contains a specific sequence that complements to 3′ half of the ligation bridge oligonucleotide C, and is flanked by a restriction enzyme recognition sequence. The 3′ terminal contains specific sequence that is self-complementary and hybridizes with the ligation sequence and the restriction sequence to form double strand DNA hairpin structure. The presence of endonuclease restriction enzyme, which recognizes the cutting site on double stranded part of oligonucleotide A and cuts off hairpin structure, allows ligation between oligonucleotides A and B held together by C. PCR is performed using primer set that span across A and B oligonucleotides to amplify the ligated product.

### Example 33

### BirA-Mediated Immobilization of a Reporter

Lectins recognizing fungal carbohydrates are conjugated to E. coli biotin ligase that specifically conjugates biotin to Avitag, a 15-aa peptide sequence. Blood samples potentially infected with fungi are added to microwells coated with the same lectin, and the wells are washed. The lectin-biotin ligase conjugate is then added to the wells, and unbound conjugate washed away. Luciferase genetically modified to be fused with the biotin ligase peptide is added to the wells, the excess is washed away, and then luciferase substrate is added. Luminescence from a given well is taken as evidence of fungal infection in the blood sample previously added to that well.

### Example 34

### Non-PCR Detection of DNA Modification

The dsDNA have both its 5′ end and/or 3′end or one 5′ end and one 3′ end covalently linked to protein, a biotin or another chemical linkage. This capping on ends confers protection of the DNA molecule from degradation by exonucleases as well as enabling the DNA to be captured or immobilized on surface. DNA is then modified with DNA modification enzyme such as endonuclease, exonuclease, nickase and helicase. These latter enzymes can be CMA for Ultra-Sensitive and Quantitative Biomarker detection as described previously. This modification exposes a new DNA end (3′ or 5′) that can be attacked by exonuclease such as Lambda exonuclease, Exonuclease I, Exonuclease III or T7 Exonuclease to create ssDNA. The ssDNA is protected by attachment of Single-Strand Binding protein. The presence of SSB can be then detected by colorimetric detection or fluorescence detection.

### Example 35

### Streptavidin Mutein Protein as CMA for Ultra-Sensitive Detection of Biomarker

In this example, the analyte or biomarker of interest is first captured by a primary ligand, such as antibody, immobilized on the surface of a solid phase, such as microplates. Streptavidin-binding peptide (SBP) tagged detection antibody specific for analyte or biomarker is then added. Streptavidin mutein protein which can bind to two SBP-tagged proteins is added such that one site of mutein protein is occupied by the SBP tagged detection antibody bound to analyte. To the other available site of streptavidin mutein protein, single-strand oligo-A and oligo-B are chemically coupled. Oligo A and B are then hybridized by single-strand Oligo C. Ligated product obtained due to ligation of oligos A and B will be then amplified using QPCR.

In another embodiment, a molecular recognition element is associated, on a nanoparticle or by conjugation, with an enzyme. The enzyme produces a compound detectable by differential mobility analysis or ion mobility spectrometry with high sensitivity. In a preferred embodiment, the enzyme is a hydrolase which liberates the compound from a less volatile precursor form, e.g. an ester or amide. In another preferred embodiment, upon light application, a photocatalyst catalyzes the formation of a more volatile form by formation or breakage of a chemical bond, for example an ester bond to form an uncharged volatile form.

The action of nucleic acid polymerases or hydrolases also can be signaled by the liberation of enzyme co-factors, or volatile compounds easily detected by DMS or gas chromatography. In particular, hydrophobic volatile compounds can be liberated from phosphate or nucleotide base groups by enzyme action.

In another embodiment, a molecular recognition element is associated with a nanoparticle or polymer also comprising a compound detectable by differential mobility analysis or ion mobility spectrometry with high sensitivity. After binding to targets and washing, the compound is liberated by the action of an enzymatic or chemical agent, or by change in pH, temperature or ionic environment, and detected by its stimulation of the activity of the detectable enzyme.

In another embodiment, a molecular recognition element is associated with a nanoparticle or polymer also comprising a compound which is a cofactor or activator for a detectable enzyme such as luciferase, phosphatase, or peroxidase. After binding to targets and washing, the compound is liberated by the action of an enzymatic or chemical agent, or by change in pH, temperature or ionic environment, and detected by its stimulation of the activity of the detectable enzyme.

The above demonstrates that avidin-coated nanoparticles can catalyze oligonucleotide ligation. This can be employed to decouple the proximity ligation process from the recognition of target proteins in PLA, reducing the background in nucleic acid amplification-based immunoassays while not being limited by the available recognition sites on the target proteins. Nanoparticle-PLA also uses PLA probes biotinylated during synthesis, avoiding the need to prepare antibody-DNA conjugates. The avidin coated nanoparticles serve as a separate module in NP-PLA whose variables such as size, geometry, and spatial distribution of avidins can be freely adjusted for optimal assay performance. In preliminary assessments, bigger nanoparticles have been found to be more detectable than smaller ones. Moreover, it has been demonstrated that melting peak based competitive PCR (mp-cPCR) is suited for the precise quantification of protein biomarkers at ultra-low levels with NP-PLA, in the above described first use of mp-cPCR in an immunoassay. Given their extremely wide applicability to protein biomarkers, ultra-sensitivity, universal design and simple preparation of the NP-PLA probes, and high precision for ultra-low-level protein quantification, the NP-PLA in combination with mp-cPCR opens a new approach to simple, sensitive and quantitative detection of protein biomarkers at ultra-low levels.

### Example 36

### Construction of Multi-Template DNA-Avidin Nanoparticles and Their Use as Immunoassay Reporters: Construction of DNA-Avidin Nanoparticles

A plasmid containing several copies of the specific template (80 bp, Sequence-5′-TGCTGCGAGAGTATTATCTTGCACCTTATGCTACCGTGATTCATCCAGTCTCATCGTGAAACAGACGTACTACTACCTG-3′) was constructed using pBC linearized using SacI and XbaI, enzymes with which the 5′ and 3′ends respectively of the commercial insert sequence are compatible. In addition, downstream of the target sequence at 3′ end, a SacI site was introduced in the synthesized oligo. Once the oligo was ligated to linearize pBC, the SacI site in pBC was eliminated. To introduce the second copy of the target sequence, the plasmid with one copy of template was linearized with SacI and XbaI enzymes and ligated to the synthesized oligo. Plasmids were transformed into recA mutant cells to ensure stability of constructs. Plasmids containing up to 7 copies of template were synthesized using iterations of this protocol. The size of plasmid with 1 copy of template was 3500 bp, and the size of the plasmid increases by 85 bp for every copy of template introduced.

Plasmid DNA (containing the desired copies of the template sequence) was diluted using deionized water to a final concentration of 1×10¹² DNA copies/ml. Avidin (Life Technologies) was pre-diluted in a separate Eppendorf tube in deionized water to a concentration such that when DNA was added to the Avidin solution, there was one Avidin molecule for every 4 bp of DNA. The concentration of Avidin was dependent on the length of plasmid containing the desired number of template copies. After chilling on ice for 30 min, the plasmid DNA was added to the Avidin such that the final volume was 1 ml. The tube was immediately vortexed for 30 sec, then mixed for 1 hour at room temperature on a rotator.

Two-arm poly(ethylene glycol)-biotin (PEG-Biotin, 10 k, Nanocs) was diluted in deionized water such that PEG-Biotin occupied 30% of available Avidin binding sites when it was added to DNA-Avidin mixture. PEG-Biotin was added to the DNA-Avidin tube and the reaction was allowed to take place for 24 hour at 4° C. on a rotator. The mixture containing DNA-Avidin nanoparticles was then filtered using an Amicon Ultra-0.5, 100 kDa membrane filter (Sigma Aldrich) to remove free Avidin and PEG-Biotin molecules. The particles were then washed four times by centrifugation with water. Purified DNA-Avidin nanoparticles obtained were then used for further analysis. Primers (Forward Primer 5′-CAGGTAGTAGTACGTCTGTT-3′, Reverse Primer 5′-GTGCTGCGAGAGTATTATCT-3′) specific for the target sequence present in the DNA-Avidin nanoparticles were designed for the use of particles in QPCR. DNA-Avidin nanoparticles can be alternately labeled with Atto-520 biotin for detection by fluorescence, or decorated with antibodies.

### Example 37

### Construction of Multi-Template DNA Avidin Nanoparticles and Their Use as Immunoassay Reporters: Use of DNA-Avidin Nanoparticles as Detectable Labels for Ultra-Sensitive Human Chorionic Gonadotropin (hCG) Detection

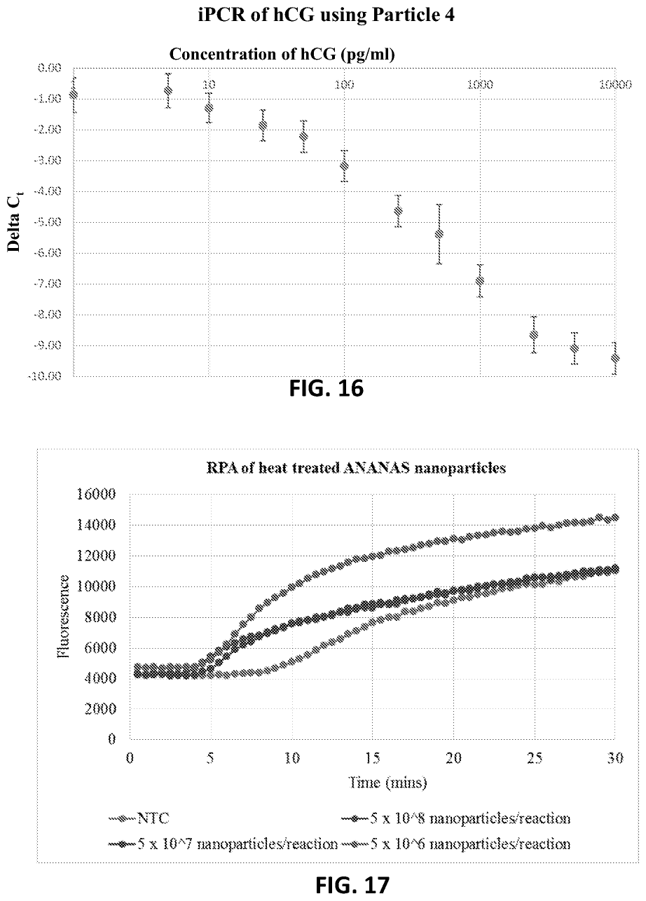

DNA-Avidin nanoparticles were used as the reporter for human chorionic gonadotropin (hCG) detection. On the bottom of 96-well microplate wells, anti-β hCG monoclonal antibody was coated. Samples potentially containing hCG were incubated 1.5 hour in the coated wells. The wells were then washed three times with PBS containing 0.1% Tween 20 and biotinylated anti-α hCG polyclonal antibody with DTT cleavable linkers (anti-α hCG polyclonal antibody biotinylated with sulfo-NHS-SS-biotin) were incubated with the wells. The wells were washed, DNA-Avidin nanoparticles were incubated in the wells, and the wells were treated with dithiothreitol (DTT). An aliquot of supernatant was then added as template to QPCR mix for amplification. Earlier amplification was taken as evidence of the presence of hCG in the sample (shown in FIG. 15 and FIG. 16). Amplification optionally may be by RPA (optionally after heating) or other method, and may be detected by hydrolysis probe, LFA of the products, or other means.

### Example 38

### DNA or RNA Replicase Such as Qβ Replicase

In this example, the analyte or biomarker of interest is first captured by a primary ligand, such as antibody, aptamer and nanobody etc., immobilized on the surface of a solid phase, such as magnetic nanoparticles or microplates. A Replicase as the CMA is covalently conjugated through a cleavable linker to a secondary ligand (MRE), such as antibody, aptamer and nanobody etc., which specifically binds to the biomarker of interest without interfering the binding between the biomarker and the primary ligand. The amount of replicase-conjugated ligand captured by the solid phase is proportional to the amount of biomarker. The captured replicase can be released as free enzymes by cleaving the cleavable linker. In the presence of its target DNA or RNA in the case of Qβ replicase, the enzyme will exponentially replicate its target. The amplified signal can be used to detect and quantify the amount of biomarker present.

### Example 39

### Use of DNA Avidin Nanoparticles to Detect Anti-Drug Antibodies (ADAs) for Biotherapeutics

In this example, the therapeutic antibody drug is first coated on a 96-well plate. Samples potentially containing anti-drug antibodies (ADAs) are incubated in the coated wells. The wells are then washed three times with PBS containing 0.1% Tween 20 and biotinylated therapeutic antibody drug with DTT cleavable linkers (antibody biotinylated with sulfo-NHS-SS-biotin) are incubated with the wells. The wells are washed, DNA-Avidin nanoparticles are incubated in the wells, and the wells are treated with dithiothreitol (DTT). An aliquot of supernatant is then added as template to qPCR mix for amplification. Earlier amplification is taken as evidence of the presence of ADAs in the sample.

### Example 40

### Use of Split-Luciferase Reporters to Detect Fusion Protein Biomarker in Tumor Cells

In this example, the capture antibody recognizing the fusion junction of protein (analyte) is first coated on a 96-well plate. Samples containing fusion protein are incubated in the coated wells. The wells are then washed three times with PBS containing 0.1% Tween 20 and the split-luciferase reporter (such as the NanoLuc Large and Small subunits, Promega) conjugated to antibodies such that one reporter subunit is conjugated to an antibody recognizing the N-terminus of fusion protein and the other reporter subunit is conjugated to an antibody recognizing the C-terminus of fusion protein. Upon formation of the 3-part complex the two reporter subunits come together in close proximity to form an active luciferase. After addition of luciferase substrate, the luciferase reporters will give luminescence signal. The signal can be used to detect and quantify the amount of fusion protein present; the higher the luminescence signal, the higher the amount of fusion protein present in the sample.

### Example 41

### Ultrasensitive Detection of Agents Used for Doping in Sports Using DNA-Avidin Nanoparticle Based iPCR

In this example, the antibody against doping agent is first coated on a 96-well plate. Samples potentially containing doping agent are incubated in the coated wells. The wells are then washed three times with PBS containing 0.1% Tween 20 and biotinylated antibody with DTT cleavable linkers (antibody biotinylated with sulfo-NHS-SS-biotin) are incubated with the wells. The wells are washed, DNA-Avidin nanoparticles are incubated in the wells, and the wells are treated with dithiothreitol (DTT). An aliquot of supernatant is then added as template to qPCR mix for amplification. Earlier amplification is taken as evidence of the presence of doping agent in the sample.

Those skilled in the art will recognize, or be able to ascertain using no more than routine experimentation, many equivalents to the specific embodiments of the invention described herein. Such equivalents are intended to be encompassed by the following claims.

1. A method for detecting an analyte in a biological sample, the method comprising:
a) providing a mixture of a biological sample potentially containing the analyte and a molecular recognition element physically coupled to a covalent modification agent, wherein the molecular recognition element is capable of specific recognition of the analyte;
b) exposing the mixture to a first set of reaction conditions, wherein the analyte and molecular recognition element can associate to form a recognition complex; and
c) upon formation of the recognition complex,
generating by use of the covalent modification agent a template complex; and
exposing the template complex to a second set of reaction conditions,
wherein the template complex is amplified to generate a highly-detectable component indicative of the presence of the analyte.

2. The method of claim 1, wherein the covalent modification agent is a helicase.

3. The method of claim 2, wherein the template complex is a single stranded DNA.

4. The method of claim 1, wherein the covalent modification agent is a DNA polymerase.

5. The method of claim 4, wherein the template complex is a double stranded DNA product.

6. The method of claim 1, wherein the covalent modification agent is a DNA glycosylase.

7. The method of claim 6, wherein the template complex is a double stranded DNA product.

8. The method of claim 1, wherein the covalent modification agent is a DNA ligase.

9. The method of claim 8, wherein the template complex is a ligated double stranded DNA product formed from two or more oligonucleotides present in the second set of reaction conditions.

10. The method of claim 9, wherein the highly-detectable component is formed by amplification of the ligated double stranded DNA product.

11. The method of claim 1, wherein the covalent modification agent is an ubiquitin protein ligase.

12. The method of claim 11, wherein the ubiquitin protein ligase is physically coupled to the molecular recognition element via a cleavable linker.

13. The method of claim 1, wherein the molecular recognition element is an antibody.

14. The method of claim 1, wherein the molecular recognition element is an aptamer.

15. The method of claim 1, wherein the analyte is immobilized to a surface of a bead.

16. The method of claim 1, wherein the analyte is immobilized to a surface of a well.

17. A method for detecting an analyte in a biological sample, the method comprising:
a) providing a mixture of a biological sample potentially containing the analyte and an antibody physically coupled to a ligase, wherein the antibody is capable of specific recognition of the analyte;
b) exposing the mixture to a first set of reaction conditions, wherein the analyte and antibody can associate to form a recognition complex; and
c) upon formation of the recognition complex,
generating by use of the ligase a ligated double stranded or single stranded DNA product; and
exposing the ligated DNA product to a second set of reaction conditions,
wherein the ligated DNA product is used to generate a highly-detectable component indicative of the presence of the analyte.

18. The method of claim 17, wherein the ligated DNA product is exposed to a polymerase.

19. The method of claim 17, wherein the ligase is physically coupled to the molecular recognition element via a cleavable linker.

20. The method of claim 17, wherein the analyte is immobilized to a surface of a bead.
